(19) United States
(12) Patent Application Publication
(10) Pub. No.: US 2022/0354806 A1
(43) Pub. Date: Nov. 10, 2022

(54) COMPOSITION FOR IMPROVING MEMORY

(71) Applicant: BGG JAPAN CO., LTD., Tokyo (JP)

(72) Inventors: Takahiro SEKIKAWA, Tokyo (JP); Yuki KIZAWA, Tokyo (JP)

(21) Appl. No.: 17/631,473

(22) PCT Filed: Jul. 31, 2020
(86) PCT No.: PCT/JP2020/029400

§ 371 Date: Jul. 25, 2022

(30) Foreign Application Priority Data
Aug. 1, 2019 (JP) 2019-141957
Nov. 1, 2019 (JP) 2019-199534

(51) Int. Cl. A61K 31/122; A61K 31/355; A61P 25/28; A61K 9/00; A23L 33/15

(52) U.S. Cl. CPC A61K 31/122; A61K 31/355; A61P 25/28; A61K 9/0056; A23L 33/15; A23V 2002/00

(57) ABSTRACT

A problem of the present application is to provide a composition for improving comprehensive memory, it has the effect of improving language memory which serves as a part of a cognitive function of a brain, and the comprehensive memory including visual memory and language memory, and the above problem is solved by providing the composition containing astaxanthins, or astaxanthins and vitamin E.

### TECHNICAL FIELD

The present disclosure relates to a composition for improving memory, and it has the effect of improving the memory which serves as a part of a cognitive function.

### BACKGROUND

Astaxanthin is used for many years as a pigment for food additives, or as a coloring agent for farmed fish. Since it is discovered that the astaxanthin has an excellent anti-oxidation effect which is 1000 times greater than that of a vitamin E, the astaxanthin is used in pharmaceuticals, health foods such as a supplement, basic cosmetics and the like.

For example, in Patent Document 1, an inspection called “Cog Health” is performed, and the inspection consists of 5 operations of pressing a button in response to a playing card displayed on a computer monitor, and compared with before ingesting the astaxanthin, after ingesting the astaxanthin, the response time of all 5 operations of “simple reaction”, “choice reaction”, “working memory”, “delayed recall” and “divided attention” is improved, and the correct answer rate in the operation of the “working memory” is improved, according to this result obtained, it is implied that the astaxanthin may improve a human cognitive function.

The “working memory” in the inspection of Patent Document 1 is a problem of judging whether a flipped playing card is the same as a previous card and pressing the button. In the inspection of Patent Document 1, the “working memory” is considered to be very short-term memory in seconds, visual memory and memory with a small amount of information.

In addition, in Patent Document 1, it is only confirmed that the cognitive function is improved after ingesting the astaxanthin compared with before ingesting the astaxanthin, and the comparison with a placebo group is not performed. In a test of comparing with the placebo group (ingesting corn oil) using the same materials and methods as in Patent Document 1, a significant difference of the improvement effect on the human cognitive function is not confirmed between the astaxanthin group and the placebo group (Non-Patent Document 1).

The vitamin E is known to be a fat-soluble vitamin that has the anti-oxidation effect and plays a role in protecting cell membranes from oxidative damage in an organism. Due to this effect, it may be expected to prevent diseases related to lifestyle diseases and aging, such as the aging caused by the oxidation of the cell membranes in vivo and arteriosclerosis caused by the oxidation of low density lipoprotein (LDL) cholesterol in blood.

However, from a viewpoint of improving the memory, there is no knowledge to confirm the synergistic effect in detail while the astaxanthin and the vitamin E are ingested together.

### EXISTING TECHNICAL DOCUMENT

### Patent Document

Patent Document 1: Japanese laid-open patent publication No. 2010-270095.

### Non-Patent Document

Non-Patent Document 1: Katagiri M, Satoh A, Tsuji S, et al. Effects of astaxanthin-rich Haematococcus pluvialis extract on cognitive function: a randomised, double-blind, placebo-controlled study. J. Clin. Biochem. Nutr. 2012; 51: 102-107.

### SUMMARY

### Problem to be Solved by the Present Disclosure

The “working memory” of Patent Document 1 is considered to be ultra-short-term memory of a very short time in seconds, visual memory and memory with a small amount of information, but the inventor et al. consider improving the ability to keep memory, the memory includes not only visual information, but also language information.

Therefore, the problem to be solved by the present disclosure is to provide a composition for improving memory, and it is capable of improving language memory in a cognitive function and comprehensive memory including visual memory and language memory.

### Scheme Used to Solve Problem

The inventor et al. conduct intensive studies in order to solve the above problem, and it is found from results that, by ingesting astaxanthins or a composition for improving memory containing astaxanthins and vitamin E, the comprehensive memory including the visual memory and the language memory may be improved, thereby the present disclosure is completed.

Namely, the present disclosure includes the following contents.

[1] A composition for improving comprehensive memory,

the composition contains the following a component (A), or a component (A) and a component (B), and the comprehensive memory includes visual memory and language memory;

the component (A): at least one selected from a group consisting of astaxanthin and an ester body of the astaxanthin and

the component (B): vitamin E.

[2] The composition according to [1], the content of the component (A) is 0.1 mass % or more and 35 mass % or less.

[3] The composition according to [1] or [2], the ester body of the astaxanthin is a monoester body and a diester body, and the mass ratio (monoester body/diester body) of the contents of the monoester body to the diester body is 2 or more and 25 or less.

[4] The composition according to any one of [1] to [3], the composition is prepared to ingest the component (A) in an amount of 3 mg or more and 15 mg or less per day.

[5] The composition according to any one of [1] to [4], the composition is prepared to ingest the component (A) in an amount of 3 mg or more and 10 mg or less per day.

[6] The composition according to any one of [1] to [5], the composition is prepared to ingest the component (A) in an amount of 8 mg or more and 10 mg or less per day.

[7] The composition according to any one of [1] to [6], the composition is prepared to ingest the component (B) in an amount of 30 mg or more and 70 mg or less per day.

[8] The composition according to any one of [1] to [7], the ingestion is oral ingestion.

[9] The composition according to any one of [1] to [8], the composition is a food.

[10] The composition according to any one of [1] to [8], the composition is a food additive.

[11] A method for improving comprehensive memory,

the method includes ingesting the following component (A) or the component (A) and the component (B), and the comprehensive memory includes visual memory and language memory;

component (A): at least one selected from a group consisting of astaxanthin and an ester body of the astaxanthin, and

component (B): vitamin E.

[12] The method according to [11], the ester body of the astaxanthin is a monoester body and a diester body, and the mass ratio (monoester body/diester body) of the daily intakes of the monoester body to the diester body is 2 or more and 25 or less.

[13] The method according to [11] or [12], the method includes ingesting at least one selected from a group consisting of astaxanthin and an ester body of the astaxanthin in an amount of 3 mg or more and 15 mg or less per day.

[14] The method according to any one of [11] to [13], the method includes ingesting the component (A) in an amount of 3 mg or more and 10 mg or less per day.

[15] The method according to any one of [11] to [14], the method includes ingesting the component (A) in an amount of 8 mg or more and 10 mg or less per day.

[16] The method according to any one of [11] to [15], the method includes ingesting the component (B) in an amount of 30 mg or more and 70 mg or less per day.

[17] The method according to any one of [11] to [16], the ingestion is oral ingestion.

### Effects of Present Disclosure

According to the present disclosure, it is possible to provide the composition having the effect of improving the comprehensive memory including the visual memory and the language memory, or the method for improving the comprehensive memory. In addition, the effect of improving the medium and long term memory in the comprehensive memory may be expected.

### BRIEF DESCRIPTION OF THE DRAWINGS

FIG. 1A is a diagram showing a score variable quantity of a cognitive function test (visual memory) before ingesting astaxanthins (ASX), after 8 weeks of ingestion, and after 12 weeks of ingestion.

FIG. 1B is a diagram showing a score variable quantity of a cognitive function test (visual memory) before ingesting astaxanthins and vitamin E (ASX+VE), after 8 weeks of ingestion, and after 12 weeks of ingestion.

FIG. 2A is a diagram showing a score variable quantity of a cognitive function test (language memory) before ingesting astaxanthins (ASX), after 8 weeks of ingestion, and after 12 weeks of ingestion.

FIG. 2B is a diagram showing a score variable quantity of a cognitive function test (language memory) before ingesting astaxanthins and vitamin E (ASX+VE), after 8 weeks of ingestion, and after 12 weeks of ingestion.

FIG. 3A is a diagram showing a score variable quantity of a cognitive function test (comprehensive memory including the visual memory and the language memory) before ingesting astaxanthins (ASX), after 8 weeks of ingestion, and after 12 weeks of ingestion.

FIG. 3B is a diagram showing a score variable quantity of a cognitive function test (comprehensive memory including the visual memory and the language memory) before ingesting astaxanthins and vitamin E (ASX+VE), after 8 weeks of ingestion, and after 12 weeks of ingestion.

FIG. 4A is a diagram showing a subjective variable quantity for such a question “did you have difficulty recalling the name of a person or thing in the past week?” before ingesting astaxanthins (ASX) and after 12 weeks of ingestion.

FIG. 4B is a diagram showing a subjective measured value for such a question “did you have difficulty recalling the names of a person or thing in the past week?” after 12 weeks of ingestion of placebo or astaxanthins and vitamin E (ASX+VE).

### DETAILED DESCRIPTION OF THE EMBODIMENTS

The present disclosure relates to a composition for improving memory containing astaxanthins or astaxanthins and vitamin E.

Astaxanthin (astaxanthine, 3,3′-dihydroxy-β,β-carotene-4,4′-dione) is a carotenoid that is the same as β-carotene in carrots and lycopene in tomatoes, and is a reddish-orange pigment substance with rich edible experience that is classified as flavonoids. The astaxanthin is widely distributed in nature, and may be seen not only in shells of crustaceans, but also on the body surfaces of red sea breams that feed on them, and in red parts of muscles of salmonidae fish.

There are stereoisomers in astaxanthin. Specifically, three stereoisomers of (3R,3′R)-astaxanthin, (3R,3′S)-astaxanthin, and (3S,3′S)-astaxanthin are known. It is generally considered that the astaxanthin derived from a natural product is mostly (3S,3′S)-astaxanthin. In the record of the description, unless otherwise stated, while it is only shown as the astaxanthin, it refers to a free body of the astaxanthin including the above three stereoisomers.

In the record of the description, while it is shown as the astaxanthins, it means at least one selected from a group consisting of the free body of the astaxanthin and an ester body of the astaxanthin. The ester body of the astaxanthin includes a monoester body and/or a diester body. Further, the monoester body and/or diester body of the astaxanthin includes the monoester body and/or diester body of each of the three stereoisomers of the astaxanthin.

In the present disclosure, the astaxanthins uses at least one selected from a group consisting of the free body and the ester body of the astaxanthin. One or both of two hydroxyls of the ester body of the astaxanthin are protected by an ester bond, so it is physically more stable than the free body, and is not easy to be oxidized and decomposed in beverages and foods or pharmaceuticals. However, while being ingested into a living body, it is considered that it may be rapidly hydrolyzed into the astaxanthin in the free body by an enzyme in the living body, and the effect is represented.

As the monoester body of the astaxanthin, monoesters esterified with a fatty acid may be listed. The fatty acid that forms the ester body of the astaxanthin is not limited in the number of carbon atoms, for example, the fatty acid having 4 or more and 30 or less carbon atoms may be listed. In addition, the fatty acid that forms the ester body of the astaxanthin may be a saturated fatty acid or an unsaturated fatty acid. As a specific example of the fatty acid that forms the ester body of the astaxanthin, it may be listed as follows: acetic acid, lauric acid, myristic acid, pentadecanoic acid, palmitic acid, palmitoleic acid, heptadecanoic acid, elaidic acid, castor oil acid, petroselinic acid, vaccenic acid, eleostearic acid, punicic acid, octadec-9,11,13-triene-4-keto acid, octadecatetraenoic acid, cis-9-eicosene acid, 5-eicosenoic acid, 5-docosenoic acid, cetoleic acid, erucic acid, 5,13-docosadienoic acid, selacholeic acid, decenoic acid, steroidal acid, dodecenoic acid, oleic acid, stearic acid, eicosapentaenoic acid, docosahexaenoic acid, linoleic acid, linolenic acid, arachidonic acid and the like.

Further, as the monoester body of the astaxanthin, it may be listed that the monoesters esterified with amino acids such as glycine and alanine; monovalent or polyvalent carboxylic acids such as acetic acid and citric acid; inorganic acids such as phosphoric acid and sulfuric acid; sugars such as glucoside; sugar fatty acids such as glycerose fatty acid and sphingolipid fatty acid; fatty acids such as glycerol fatty acid; and glycerophosphoric acid and the like. As the monoester body of the astaxanthin, salts of the monoesters are also included.

As the diester body of the astaxanthin, the same or different acid-esterified diesters selected from a group consisting of the fatty acid, the amino acid, the monovalent or polyvalent carboxylic acid, the inorganic acid, the sugar, the sugar fatty acid, the fatty acid and the glycerophosphoric acid may be listed. It should be noted that salts of the diesters are also included in the case that it may be considered.

In the present disclosure, the astaxanthins refer to astaxanthin derived from a natural product or astaxanthin obtained by synthesis. As the astaxanthin derived from the natural product, for example, the astaxanthin obtained from shells, eggs and organs of crustaceans such as a shrimp, a krill, and a crab, skins and eggs of various fish and shellfish, algae such as green alga Haematococcus, yeasts such as Phaffia rhodozyma, bacteria such as marine bacteria Paracoccus, and seed plants such as Adonis and Goldilocks may be listed. Naturally derived extracts and chemical synthetics are commercially available, and readily available.

The astaxanthins may be obtained, for example, by culturing astaxanthins-producing microorganisms such as the Phaffia rhodozyma, the green alga Haematococcus, and the marine bacteria Paracoccus in a suitable culture medium according to a known method. The green alga Haematococcus is the most preferable in view of the easiness of culture or extraction, the highest concentration of the astaxanthin, and the level of productivity.

The green alga is not particularly limited as long as it is green alga capable of producing the astaxanthins. For example, unicellular algae belonging to a Haematococcus genus are preferably used. As the preferred green alga, H. pluvialis, H. lacustris, Haematococcus capensis (H. capensis), Haematococcus droebakensi (H. droebakensi), H. zimbabwiensis and the like may be listed.

The astaxanthins obtained by the green alga Haematococcus is mostly the monoester body bonded with the fatty acid on one hydroxyl of the astaxanthin, and secondly, the diester body bonded with the fatty acids at both ends, and the astaxanthins existing in the unmodified free body is present in a very small proportion. The astaxanthins obtained from the Phaffia rhodozyma mostly exists in the unmodified free body. In addition, the astaxanthins obtained from the marine bacteria Paracoccus also mostly exists in the unmodified free body.

In the present disclosure, the astaxanthins-containing extract is produced as follows. The astaxanthins-producing microorganisms are cultured in a nutrient medium, and then an astaxanthin-containing oily substance is extracted. The means for extracting the astaxanthin-containing oily substance is not particularly limited, and the means commonly used by those skilled in the art may be used. For example, for the cultured microorganisms for producing the astaxanthins, the extraction is performed by solvent suspension, drying, mechanical crushing, pressing, supercritical extraction and other operations. These extraction operations may be performed individually or in combination.

As a solvent for the extraction, an organic solvent such as chloroform, hexane, acetone, methanol, and ethanol may be used. A drying method may be performed according to conventional methods such as shelf-type drying, fluidized bed drying, flash drying, and spray drying. A method of mechanical pulverization may be either a wet method or a dry method, and may be performed by a bead mill, a roll mill, a hammer mill, a jet mill, a pin mill and the like. A pressing method may be performed according to a conventional method. The supercritical extraction shapes the astaxanthins-producing microorganisms that are treated and pulverized into a granular shape according to a conventional method, and carbon dioxide in a supercritical state or carbon dioxide in a state near a supercritical point is extracted through a layer filled with the granules, and then the carbon dioxide is removed under a reduced pressure to obtain an extract. In order to improve the extraction efficiency from the granules, the higher unsaturated fatty acid, glycerol, alcohol, water and the like may be added.

In the case that the solvent is used in the extraction, after the extraction, the solvent is removed by the means commonly used by those skilled in the art. Further, in the case that it is desired to remove the solvent, a molecular distillation device and the like may be used. The astaxanthins-containing extract may be further purified by separation column or lipase decomposition as desired.

The content of the astaxanthins in the composition is usually 0.1 mass % or more, preferably 0.5 mass % or more, more preferably 1 mass % or more, and usually 35 mass % or less, preferably 30 mass % or less, more preferably 25 mass % or less, and these upper and lower limits may be any combinations. In addition, the content of the astaxanthins is usually 0.1 mass % or more and 35 mass % or less, preferably 0.5 mass % or more and 30 mass % or less, and more preferably 1 mass % or more and 25 mass % or less.

The ratio of the monoester body in the astaxanthins contained in the composition is usually 40 mass % or more, preferably 50 mass % or more, more preferably 60 mass % or more, further preferably 70 mass % or more, and especially preferably 80 mass % or more. The upper limit of the content of the monoester body in the astaxanthins is not particularly limited, but is usually 100 mass % or less. For the ratio of the monoester body in the astaxanthins, these upper and lower limits may be any combinations. The ratio of the monoester body in the astaxanthins contained in the composition is 40 mass % or more and 100 mass % or less, preferably 50 mass % or more and 100 mass % or less, more preferably 60 mass % or more and 100 mass % or less, further preferably 70 mass % or more and 100 mass % or less, and especially preferably 80 mass % or more and 100 mass % or less.

The ratio of the diester body in the astaxanthins contained in the composition is usually 40 mass % or less, preferably 35 mass % or less, more preferably 30 mass % or less, further preferably 25 mass % or less, and especially preferably 20 mass % or less. The lower limit of the ratio of the diester body in the astaxanthins contained in the composition is not particularly limited, but is usually 0 mass % or more. In addition, for the ratio of the diester body in the astaxanthins, these upper and lower limits may be any combinations. The ratio of the diester body in the astaxanthins contained in the composition is usually 0 mass % or more and 40mass % or less, preferably 0 mass % or more and 35 mass % or less, more preferably 0 mass % or more and 30 mass % or less, further preferably 0 mass % or more and 25 mass % or less, and especially preferably 0 mass % or more and 20 mass % or less.

The mass ratio of the monoester body to the diester body in the astaxanthins contained in the composition (monoester body/diester body) is usually 2 or more, preferably 3 or more, more preferably 4 or more, further preferably 6 or more, and especially preferably 8 or more, and usually 25 or less, preferably 20 or less, more preferably 15 or less, further preferably 13 or less, and especially preferably 10 or less. These upper and lower limits may be any combinations. For the mass ratio of the monoester body to the diester body (monoester body/diester body) in the astaxanthins contained in the composition is usually 2 or more and 25 or less, preferably 3 or more and 20 or less, more preferably 4 or more and 15 or less, further preferably 6 or more and 13 or less, and especially preferably 8 or more and 10 or less. It is preferable that the mass ratio of the monoester body to the diester body in the astaxanthins contained in the composition is within the above range in view of aspects that both the stability in the composition and the effect in vivo may be achieved.

The daily intake of the astaxanthins is usually 3 mg or more, preferably 5 mg or more, more preferably 8 mg or more, and especially preferably 9 mg or more, and usually 15 mg or less, preferably 13 mg or less, more preferably 11 mg or less, and especially preferably 10 mg or less, and these upper and lower limits may be combined arbitrarily. The daily intake of the astaxanthins is usually 3 mg or more and 15 mg or less, preferably 3 mg or more and 10 mg or less, more preferably 8 mg or more and 10 mg or less, and especially preferably 9 mg or more and 10 mg or less. The daily intake of the astaxanthins is preferably within the above range in order to sufficiently obtain the effects of the present disclosure.

The composition of the present disclosure may be a composition containing the above astaxanthins, or may be a composition containing astaxanthins and vitamin E.

In this case, it is sufficient to mix the vitamin E with the above such astaxanthins. It should be noted that a mixing sequence of the astaxanthins and the vitamin E is not limited. The preferable content of the astaxanthins in the composition containing the astaxanthins and vitamin E is as described above.

The main physiological effect of the vitamin E is the anti-oxidation effect, and it has the effect of protecting the cell membrane from the oxidative damage in the living body. In the present disclosure, the vitamin E is ingested together with the astaxanthins, and functions as an activator for improving the action effect of the astaxanthins. The vitamin E is a fat-soluble vitamin, and there are 8 chemically different compounds depending on the position or presence or absence of a methyl in a cyclic moiety in the structure. The vitamin E includes 4 tocopherols and 4 tocotrienols starting with α-, β-, γ- and δ-, respectively. Herein, the tocotrienols are preferably used. The tocotrienols may have the higher anti-oxidation effect than the tocopherols.

In the present disclosure, the vitamin E may be a vitamin E derived from a natural product or a vitamin E obtained by synthesis. The tocopherol may be obtained from vegetable oil such as sunflower seed oil, cottonseed oil, safflower oil, rice bran oil, corn oil, rapeseed oil, soybean oil, palm oil, olive oil, perilla oil, linseed oil, and sesame oil. The tocotrienol may be obtained from rice, barley, wheat, rye, oil palm (palm oil) and annatto seeds. Since all types of α-, β-, γ-, and δ-tocotrienol are contained in a large amount, the tocotrienol obtained from oil palm (palm oil) is preferably used.

The content of the vitamin E in the composition containing the astaxanthins and vitamin E is usually 10 mass % or more, preferably 14 mass % or more, and more preferably 16 mass % or more, and usually 50 mass % or less, preferably 40 mass % or less, and more preferably 30 mass % or less, and these upper and lower limits may be any combinations. In addition, the content of the vitamin E is usually 10 mass % or more and 50 mass % or less, preferably 14 mass % or more and 40 mass % or less, and more preferably, 16 mass % or more and 30 mass % or less.

The daily intake of the vitamin E is usually 30 mg or more, preferably 35 mg or more, more preferably 40 mg or more, and especially preferably 45 mg or more, and usually 70 mg or less, preferably 65 mg or less, more preferably 60 mg or less, and especially preferable 55 mg or less, and these upper limits and lower limits may be combined arbitrarily. The daily intake of the vitamin E is usually 30 mg or more and 70 mg or less, preferably 35 mg or more and 65 mg or less, more preferably 40 mg or more and 60 mg or less, and especially preferably 45 mg or more and 55 mg or less.

In the description, the daily intake refers to a daily intake of an adult.

The composition for improving the memory in the description is prepared in a mixed amount containing the astaxanthins, so that the daily intake is within a prescribed range by one to several intakes.

It should be noted that while a raw material containing the astaxanthins is used (concentration, and intake amount), it is calculated on the basis of the amount of the component itself in the raw material. In addition, the content (concentration, and intake amount) of the astaxanthins is based on a value obtained by converting the mass of the ester body or salt to the mass of the equimolar free body while the astaxanthin has the ester body or salt.

Another mode of the present disclosure is a method for improving the memory by ingesting the astaxanthins or the astaxanthins and vitamin E. The content of the astaxanthins or the astaxanthins and vitamin E, and the daily intake of the astaxanthins or the astaxanthins and vitamin E are as described above. As an ingestion method, for example, the oral ingestion may be listed. In the method of the present disclosure, the vitamin E, and the free body, the monoester body and/or the diester body of the astaxanthin which is used as the astaxanthins are ingested respectively independently. In the method of the present disclosure, in order to obtain the sufficient effects, it is preferable to continuously ingest the astaxanthins or the astaxanthins and vitamin E, usually for 4 weeks or more, preferably 8 weeks or more, and especially preferably 12 weeks or more.

In the present disclosure, as long as the effect of the present disclosure is obtained by containing the astaxanthins or the astaxanthins and vitamin E, the shape of the composition is not particularly limited, for example, it may be used in arbitrary shapes of liquid, paste, gel, solid and the like. In addition, a usage form of the composition is not particularly limited as long as the effects of the present disclosure may be obtained, and it may be used in any form such as beverage, syrup, cream, jelly, powder, granule, tablet, soft capsule, and hard capsule. In particular, the present disclosure is preferably used in the form of the soft capsule. In addition, the composition for improving the memory of the present disclosure may contain other arbitrary components, such as a sweetener, a flavor, an excipient, and a coloring agent, in the range which does not hinder the effect of the present disclosure. In the present disclosure, the composition for improving the memory may be produced in the form exemplified by a known method. In the present disclosure, dried products such as the Phaffia rhodozyma, the green algae Haematococcus, and marine bacteria Paracoccus and their crushed products may also be used directly together with the vitamin E. The astaxanthins may be used alone in the form of the astaxanthins-containing extract obtained by the above extraction method and the powder or aqueous solution containing the same, or used together with the vitamin E.

In the case that the composition is prepared as the soft capsule, the mass of a film relative to the entire soft capsule is usually 35 mass % or more and 50 mass % or less, preferably 40 mass % or more and 45 mass % or less.

The composition of the present disclosure may be used as a pharmaceutical ingredient, a food ingredient, a food additive, and the like.

The composition of the present disclosure may be used as a capsule, an aqueous preparation, a suspension agent, an emulsion agent, a syrup agent, an elixir, an injection agent, a suppository, an inhalant, a nasal preparation, a transdermal preparation and the like.

As the food, it is produced as a supplement, a solid food, a liquid food, a beverage and the like by conventional methods, but is not limited to these. Here, “food” refers to beverages and foods, and includes all beverages and foods in the form of oral use. As the food, it may be a functional food. The functional food refers to, for example, a health food, health accessory food, a food for patients, a nutritional accessory food, or a health functional food (a specific health food, a nutritional functional food, and a functional display food) prescribed by the Ministry of Health, Labor and Welfare which have the superior physiological properties compared to the common foods. The composition of the present disclosure may be used as a component of the health food, the health accessory food, the food for patients, the nutritional accessory food, the specific health food, the nutritional functional food, and the functional display food.

Further, the display attached to the above functional food includes, for example, a display approved for the health functional food prescribed by the Ministry of Health, Labor and Welfare, or a display intended to improve the memory. Specifically, these displays are attached to a packaging container of the functional food, for example.

In the present disclosure, the visual memory, the language memory, and the comprehensive memory are included in the concept of a brain cognitive function. The cognitive function is a series of operations for recognizing stimuli from internal and external environments, and is defined as a comprehensive ability as intellectual activities such as perception, judgment, attention, memory, thinking, and language comprehension.

The cognitive function may be evaluated by various measurement methods such as a neuropsychological test and a neurophysiological test. As the neuropsychological test, Cog Health, Cognitrax Wexler adult intelligence test, Stanford Binet intelligence test, VPTA standard advanced visual perception test, SPTA standard advanced action test, Wexler memory test, CATCAS standard attention inspection method standard positive evaluation method, D-CAT attention function screening test, Hamamatsu advanced brain function scale, Neostrup inspection, Hasegawa dementia scale, N-type mental function inspection, COGNISTAT, MEDE multifaceted initial dementia judgment inspection, NS dementia symptom inspection, TAIS, MMSE and the like may be listed. As the neurophysiological test, there is an event-related potential of a brain wave and the like, and as the event-related potential, concomitant negative variation (CNV), P1-N1-P2, NA, Nd, N2b, P300, MMN, N400 and the like may be listed. In the medicine, the cognitive function may be evaluated by the activity measurement of the advanced functions of the brain based on functional magnetic resonance imaging (fMRI), single photon emission computed tomography (SPECT), light topography and the like. In addition, in the general public, it may also be evaluated by a series of video game software and the like known as so-called brain exercise. In addition, the cognitive function may also be evaluated by physical measurement such as investigation of light reaction time.

For example, as in a test example described later, it is preferable to measure the cognitive function by an examination including a test performed according to an instruction displayed in characters on a personal computer screen. Therefore, the present disclosure is preferably applicable to a person who may understand the language. In terms of ages, it is preferable to apply the present disclosure to a person over 5 years old. It is generally believed that the cognitive function increases with the age from birth, peaks around the age of 20 years old, and then declines. The present disclosure is more preferably applied to a person over 40 years old, and especially preferably applied to a person over 50 years old. The upper limit of the age of an application object is not particularly limited, and is usually under 100 years old.

The present disclosure is preferably applicable to a healthy person. In the present disclosure, “healthy person” refers to a person who is not diagnosed with diseases such as memory impairment, and is not a person who is hospitalized or recuperates from illness, but a person who lives a normal life.

In the present disclosure, the memory is an ability that relates to the memory at least based on the language comprehension within the cognitive function defined above (the comprehensive abilities as the intellectual activities such as the perception, judgment, attention, memory, thinking, and language comprehension).

In the description, from the viewpoint of the memory, the memory may be classified into the visual memory, the language memory and the like, and from the viewpoint of the length of the time of the memory, it may be classified into the ultra-short-term memory, the short-term memory, and the medium and long-term memory. In the present disclosure, the memory includes the action memory (working memory). In the description, the comprehensive memory includes the visual memory and the language memory.

In the description, the visual memory is memory about visual-spatial information such as graphics and colors, for example. In addition, in the description, the language memory is memory about language information such as the concept of a character, and the meaning of a number, for example.

In the description, the ultra-short-term memory refers to memory that disappears while certain information is recognized, for example, within less than 30 seconds, the short-term memory refers to memory that disappears while certain information is recognized after a certain period of time (for example, less than 5 minutes), and the medium and long-term memory refers to memory in which a part of the recognized information remains after a certain period of time (for example, more than 5 minutes). In the description, recalling the name of a person or thing means recalling information memorized before the above cognitive function test, and a period of the memory is included in the medium and long-term memory which is longer than the short-term memory.

In the description, the action memory (working memory) is a concept used while the memory is used as a tool in order to complete the problem, not for the purpose of the memory itself like the short-term memory, and it is a storage system which may be temporarily memorized and recalled for the short-term memory. While complex work such as calculation, and reading comprehension is performed in the mind, the required information needs to be memorized somewhere. Such memory is the working memory (action memory). Namely, the action memory is something like a memo, and has three functions of writing, saving, and checking necessary information afterwards.

It is generally recognized that the action memory is composed of a phonological loop that processes voice and language information, a visuospatial sketchpad that processes visuospatial information, and a central executive system that controls both and undertakes the activities for executing the problem. The information is taken in the phonological loop or the visuospatial sketchpad, and the central executive system adjusts which memory or operation to pay more attention to while observing the progress of the problem execution.

The cognitive function inspection known as “Cognitrax” used in a test example of the present disclosure is a cognitive function inspection technology developed by CNS Vital Signs Company in the United States. Based on existing cognitive function inspection, a computer is used on the network to implement the inspection. “Cognitrax” may measure a wide range of functional areas such as the memory, the attention, the processing speed, and the executive function, and results may be displayed by numerical value and comparison of an age standard value. “Cognitrax” provides 10 types of tests, which may be selected according to the purpose of the inspection, and may detect changes in the memory and the cognitive function by monitoring an individual value over time. The 10 tests include inspection items of language memory, visual memory, finger tapping, Symbol Degit Coding (SDC), Stroop, attention shift, continuous processing, expression recognition, logical thinking, and 4 parts of continuous processing.

The language memory test refers to the following test: memorizing 15 words displayed on a screen for two seconds each, immediately thereafter finding the memorized word from 30 words that also contain new words, and after checking all of them, finding the memorized words from the 30 words that also contain the new words again.

The visual memory test refers to the following test: memorizing 15 patterns that are displayed on a screen for two seconds each, immediately thereafter finding the memorized figures from 30 patterns that also contain new patterns, and after checking all of them, finding the memorized patterns from the 30 patterns that also contain the nevv patterns again.

The finger tapping test refers to the following test: using an index finger to tap a space key as fast as possible for 10 seconds, 3 times with a right hand, and 3 times with a left hand, for a total of 6 times.

The SDC test refers to the following test: as in a sample screen 1 below, an upper part of the screen displays 8 figures corresponding to a legend containing 8 symbols, and a lower part of the screen displays 8 legends and 8 blank columns in a transposed order, a subject inputs the numbers corresponding to the legends into the blank columns at the bottom of the screen.

The Stroop test is a t st consisting of the following 3 parts.

Part 1: if the characters appear in the screen, the space key is pressed as early as possible.

Part 2: the red, yellow, blue and green characters are displayed in the screen as color characters. If the color of the characters is consistent with the meaning of the characters, the space key is pressed.

Part 3: the space key is pressed only while the color of the characters is not consistent with the meaning of the characters.

The attention shift test refers to the following test: 3 pictures are given, 1 picture is displayed on an upper part of the screen, 2 pictures are displayed on a lower part of the screen, and the picture on the lower part of the screen is selected according to a predetermined rule to snatch the picture on the upper part of the screen. Rule: a picture with the same “shape” is selected, or a picture with the same “color” is selected.

The continuous processing test refers to the following test: the space key is pressed only while “B” is displayed among the characters randomly displayed on the screen.

The facial expression recognition test refers to the following test: the space key is pressed in the case that the facial expression displayed on the screen is consistent with the language meaning written under the face.

The logical thinking test refers to the following test: as in a sample screen 2 below, a pattern is displayed in the center of the screen, it is divided into 4 partitions, there are diagrams in 3 partitions, and 1 partition is a blank column, a diagram which should fill in the blank partition is inferred, and a figure corresponding to the diagram in the lower part of the screen is selected from candidate diagrams displayed in the lower part of the screen.

The 4 parts of the continuous processing tests is composed of the following 4 parts. The 4 parts of the, continuous processing tests may measure the action memory and sustained attention.

Part 1: simple reaction speed: a test of pressing the space key as early as possible while displaying graphics (a square, a circle and the like) on the screen.

Part 2: continuous processing test: a test of pressing the space key as early as possible while a specified particular graphic (color and shape) is displayed in the randomly displayed graphics.

Part 3: checking the memory of the previous one diagram: a test of pressing the space key as early as possible while the same graphic (color and shape) as before is displayed in the randomly displayed graphics.

Part 4: checking the memory of the previous two diagrams: a test of pressing the space key as early as possible while the same graphic (color and shape) as the previous two diagrams is displayed in the randomly displayed graphics.

“Cognitrax” evaluates the items of neurocognitive index (NCI), comprehensive memory, language memory, visual memory, cognitive function speed, response time, comprehensive attention, cognitive flexibility, processing speed, executive function, social cognition, logical thinking, working memory, sustained attention, pure attention, and action speed through the above 10 tests. In the evaluation of the “Cognitrax” inspection, the comprehensive memory is expressed as the sum of scores of the language memory and the visual memory.

A calculation method for the scores of the comprehensive memory is described.

The language memory test refers to the following test: memorizing 15 words that are displayed on the screen for two seconds each, and then immediately finding the memorized words (immediate memory scores) from 30 words that also contain new words, and after checking all of them, finding the memorized words (delayed memory scores) from the 30 words that also contain the new words again. The visual memory test is a test that turns the words memorized in the language memory test into the graphics, and the process is the same as the language memory test.

The comprehensive memory is determined from a correct number of the language memory tests and visual memory tests, and converted to a standardized score. The standardized score is a standardized score in normal distribution, obtained from a measured value, with a mean value of 100 and a standard deviation value of 15. If it is 1 SD better than the mean value of people of the same age, the standardized score is 115.

As described above, in the inspection of the visual memory in “Cognitrax”, 15 graphics are continuously displayed, and in the inspection of the language memory, 15 words are continuously displayed, and it is inspected whether they are memorized. The comprehensive memory in “Cognitrax” has an extraordinarily large amount of information compared to the “working memory” in Patent Document 1, the pre-memory time is longer in minutes, and not only visual information is inspected, but also the memory related to language information is inspected.

It is speculated by the inventor et al that in the brain of the person who ingests the astaxanthins or the astaxanthins and vitamin E, the astaxanthins or the astaxanthins and vitamin E have the anti-oxidation effect, neurological protective effect and anti-inflammatory effect, thus the improvement effect of the memory such as the visual memory and the language memory may be obtained.

### Embodiment

Hereinafter, the present disclosure is described more specifically based on embodiments, but the present disclosure is not limited to these embodiments. It should be noted that unless otherwise specified, the unit is a mass basis.

<Preparation of Astaxanthins-Containing Composition>

Haematococcus pluvialis is cultured at 25° C. under a light irradiation condition with aeration of a gas containing 3% CO₂ while nutrient stress (lack in nitrogen source) is applied, and it is induced. Induced cells are crushed by a beads beater, and the oily substance containing the astaxanthin is extracted with ethanol. A crude extract is concentrated under a reduced pressure and the ethanol is distilled off, to prepare an ethanol extract containing 10 mass % of the astaxanthin in terms of the free body.

In addition, the induced cells are pulverized in the same manner as above, and then formed into a granule shape, and extracted through filling the carbon dioxide in a supercritical state or carbon dioxide in a state near the supercritical point in the layers of the granules, and then the carbon dioxide is removed under the reduced pressure, to prepare a supercritical extract.

### MANUFACTURING EXAMPLE

Manufacturing Example 1 is obtained by mixing 90 mg of the ethanol extract obtained by the above method and 70 mg of “TheraPrimE™ palm tocotrienol 92%” (manufactured by BGG Japan Company).

Manufacturing Example 2 is obtained by mixing 90 mg of the supercritical extract obtained by the above method and 70 mg of “TheraPrimE™ palm tocotrienol 92%” (manufactured by BGG Japan Company).

Manufacturing Examples 1 and 2 are prepared into the soft capsules, and are used as Manufacturing Examples 3 to 8 of the compositions shown in Table 1 respectively. Manufacturing Examples 3 to 8 contain the astaxanthins (ASX).

On the other hand, as shown in Table 2, the astaxanthins in Manufacturing Examples 3 to 8 is mixed with the vitamin E respectively to prepare Manufacturing Examples 9 to 14. Manufacturing Examples 9 to 14 contain an astaxanthins and vitamin E group (ASX+VE).

<Preparation of Test Food>

Manufacturing Example 3 is used as a test food for the astaxanthins (ASX). A grain of the test food per day contains 9 mg (3.26 mass %) of the astaxanthins.

Manufacturing Example 9 is used as a test food for the astaxanthins and vitamin E group (ASX+VE). A grain of the test food per day contains 9 mg (3.26 mass %) of the astaxanthin and 50 mg (18.1 mass %) of the tocotrienol.

89.3 mass % of the monoester body and 9.4 mass % of the diester body are contained in the astaxanthins.

In addition, 70 mL of a medium chain fatty acid (MCT) is prepared as the soft capsule, and used as a test food of a placebo group.

TABLE 1

| | | | Content of astaxanthins relative to whole (%) | | | | | Proportion of astaxanthins (%) | | |
|---|---|---|---|---|---|---|---|---|---|---|
| | | Manufacturing | Free | Monoester | Diester | | Content | Free | Monoester | Diester |
| | | example | body | body | body | Total | (mg) | body | body | body |
| Astaxanthins ethanol extract | | 1 | 0.15 | 10.28 | 1.08 | 11.51 | — | 1.30 | 89.30 | 9.40 |
| Astaxanthins supercritical extract | | 2 | 0.14 | 9.99 | 2.26 | 12.39 | — | 1.16 | 80.60 | 18.24 |
| Soft | Prepared by | 3 | 0.04 | 2.91 | 0.31 | 3.26 | 9 | 1.30 | 89.30 | 9.40 |
| capsule | Manufacturing | 4 | 0.03 | 1.94 | 0.20 | 2.17 | 6 | 1.30 | 89.30 | 9.40 |
| | Example 1 | 5 | 0.01 | 0.97 | 0.10 | 1.09 | 3 | 1.30 | 89.30 | 9.40 |
| | Prepared by | 6 | 0.04 | 2.63 | 0.59 | 3.26 | 9 | 1.16 | 80.60 | 18.24 |
| | Manufacturing | 7 | 0.03 | 1.75 | 0.40 | 2.17 | 6 | 1.16 | 80.60 | 18.24 |
| | Example 2 | 8 | 0.01 | 0.88 | 0.20 | 1.09 | 3 | 1.16 | 80.60 | 18.24 |

TABLE 2

| Manufacturing | ASX content | | VE content | | Proportion of astaxanthins (%) | | |
|---|---|---|---|---|---|---|---|
| example | % | mg | % | mg | Free body | Monoester | Diester |
| 9 | 3.26 | 9 | 18.1 | 50 | 1.30 | 89.30 | 9.40 |
| 10 | 2.17 | 6 | 10.9 | 30 | 1.30 | 89.30 | 9.40 |
| 11 | 1.09 | 3 | 25.4 | 70 | 1.30 | 89.30 | 9.40 |
| 12 | 3.26 | 9 | 18.1 | 50 | 1.16 | 80.60 | 18.24 |
| 13 | 2.17 | 6 | 10.9 | 30 | 1.16 | 80.60 | 18.24 |
| 14 | 1.09 | 3 | 25.4 | 70 | 1.16 | 80.60 | 18.24 |

<Subject>

In the evaluation test of the astaxanthins, the subjects are 34 healthy Japanese adult men and women aged 41 to 71 years old (astaxanthins group: n=16, and placebo group: n=18).

In the evaluation test of the astaxanthins and vitamin E, the subjects are 36 healthy Japanese adult men and women aged 41 to 75 years old (astaxanthins and vitamin E group: n=18, and placebo group: n=18).

<Intake of Test Food>

The subjects in the astaxanthins group ingest 1 soft capsule of Manufacturing Example 3 during or after breakfast for 12 weeks continuously. The subjects in the astaxanthins and vitamin E group ingest 1 soft capsule of Manufacturing Example 9 during or after breakfast for 12 weeks continuously. On the other hand, the subjects in the placebo group ingest 1 soft capsule of the medium chain fatty acid (MCT) during or after breakfast for 12 weeks continuously.

<Statistical Analysis>

In this test, a total of three inspections are performed at the time of test registration (before ingestion of the test food), 8 weeks after the start of ingestion, and 12 weeks after the start of ingestion, and the cognitive function is measured. Measurement data at the time of test registration (before ingestion of the test food) is used as a baseline, and a value obtained by subtracting the baseline from the measurement value from the start of ingestion to each checkpoint is used as a variation (after Δ8 weeks, and after Δ12 weeks). As the background of the subjects, age, mini-mental state examination (MMSE), IgE (radio immuno sorbent test (RIST)), and visual acuity are demographically counted, and the inter-group comparison is made by using Welch's t-test. The baseline and variation in the cognitive function test are expressed as mean and standard deviation, and the inter-group comparison is made by using Welch's t-test. About subjective symptoms, the inter-group comparison is performed between the base line and the measurement values after 8 and 12 weeks of ingestion by using Mann-Whitney's U test respectively. All statistical analyses are performed by a two-sided test, and a significance level is set at 5%. Software uses SPSS Ver.23.0 for Windows version (manufactured by IBM joint-stock company, Japan). The multiplicity caused by multi-item and multi-time is not considered, and the significance probability is not adjusted.

### TEST EXAMPLE 1

Cognitive Function Test “Cognitrax”

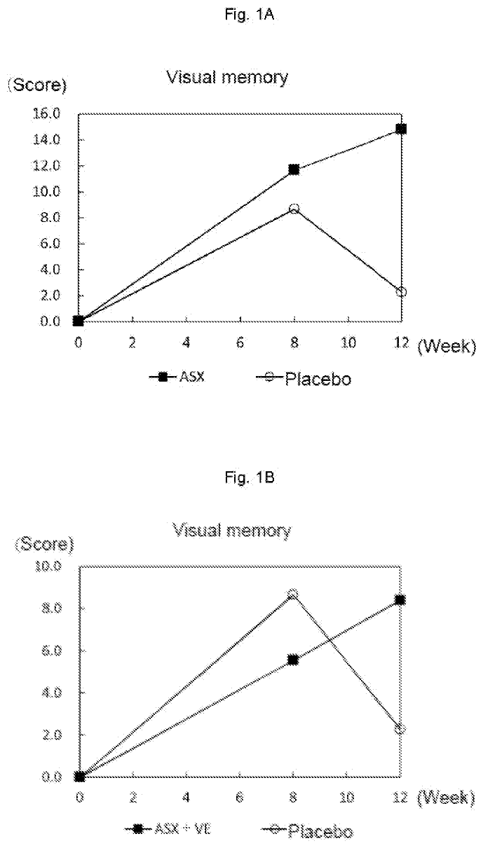

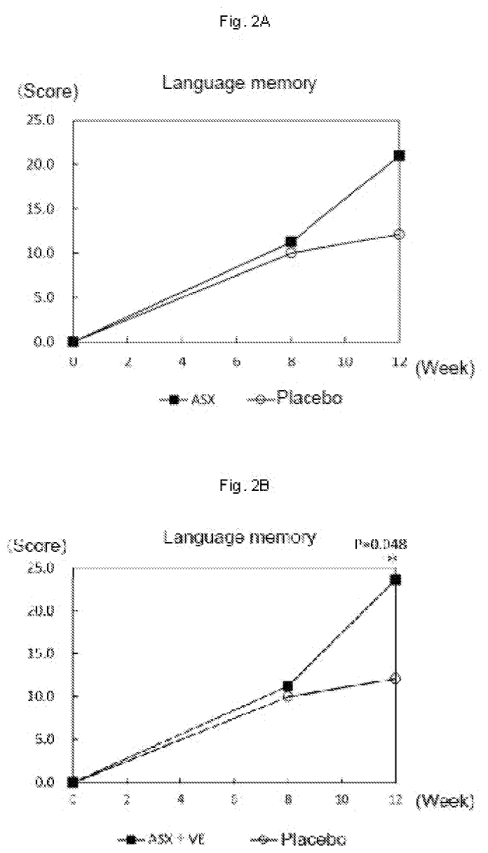

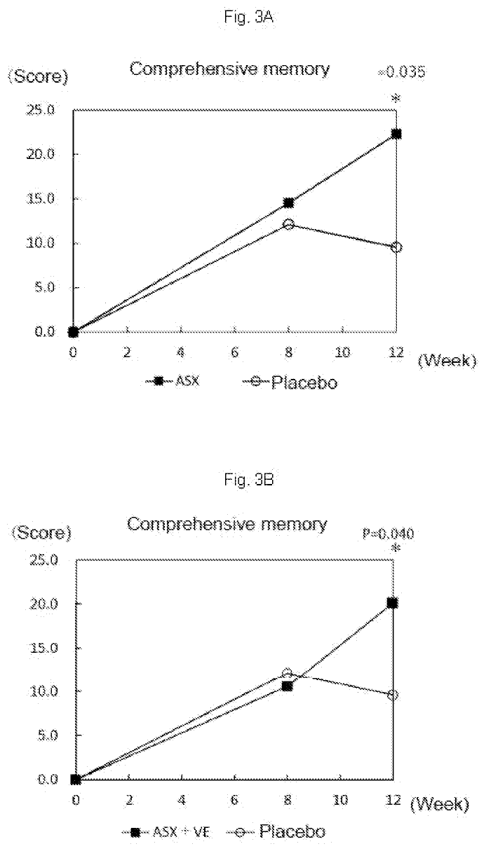

In Test Example 1, the cognitive functions before the ingestion of the test food, after 8 weeks of the ingestion, and after 12 weeks of the ingestion are measured by “Cognitrax”. In test results, about the comprehensive memory calculated from the visual memory, the language memory and their sums which are the test items related to the memory, the variations are respectively shown in FIGS. 1 to 3. It may be judged from FIG. 2 that: compared with the placebo group, the language memory (variation) of the astaxanthins and vitamin E group (ASX+VE) is significantly improved after 12 weeks of ingestion. It may be judged from FIG. 3 that: compared with the placebo group, the comprehensive memory (variation) of the astaxanthins (ASX) or astaxanthins and vitamin E group (ASX+VE) is significantly improved after 12 weeks of ingestion.

### TEST EXAMPLE 2

Subjective Questionnaire Survey Related to Memory

In Test Example 2, before ingestion of the test food, after 8 weeks of ingestion, and after 12 weeks of ingestion, the subjective questionnaire survey related to memory is administered. Question items of the subjective questionnaire survey are shown in Table 3 below, and the subjectivity is evaluated by using a Likert scale method. The evaluation is performed in 6 grades, a numerical value is lower, and the subjectivity related to the memory is better. An evaluation benchmark is as follows. 1. “Not at all”, 2. “Almost not”, 3. “Not quite”, 4. “Slightly”, 5. “Quite”, 6. “Very”.

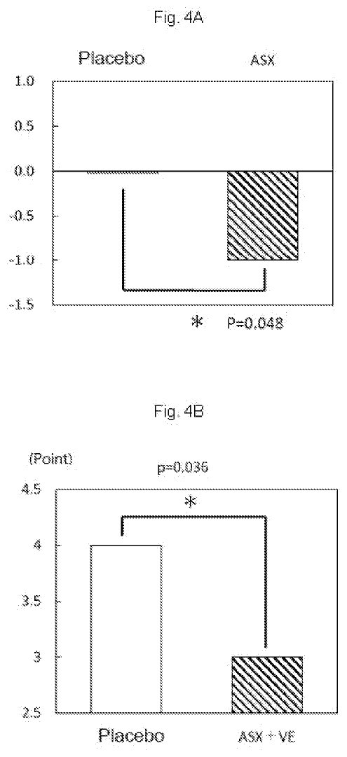

Results of the questionnaire survey are shown in FIG. 4. In such a question “did you have difficulty recalling the name of a person or thing in the past week?”, the measurement values of the astaxanthins (ASX), or astaxanthins and vitamin E group (ASX+VE) are significantly reduced after 12 weeks of ingestion compared with the placebo group. Recalling the name of a person or thing refers to recalling information memorized before this cognitive function test, and a memorized period is equivalent to the medium and long-term memory longer than the short-term memory. It may be judged that: the medium and long-term memory is improved by ingesting the test food.

TABLE 3

Question item
Did you often forget things in the past week?
Did you feel memory loss in the past week?
Did you have a moment that you couldn't remember what you had heard in the past week?
Did you have difficulty recalling the name of a person or thing in the past week?
Did you forget anything in the past week?
Did you feel chronic fatigue in the past week?
Did you feel eyestrain in the past week?
Did you feel pain in your neck and shoulders in the past week?
Did you have a moment of depression in the past week?
Did you feel discomfort in your waist in the past week?
Did you have difficulty getting up from the floor or chair in the past week?
Did your knee hurt while you squatted down or stood up in the past week?
Did your knee hurt while you went up and down stairs in the past week?

What is claimed is:

1. A composition for improving comprehensive memory, wherein,
the composition comprises a component (A), or a component (A) and a component (B), and the comprehensive memory comprises visual memory and language memory;
the component (A): at least one selected from a group consisting of astaxanthin and an ester body of the astaxanthin, and
the component (B): vitamin E.

2. The composition for improving the comprehensive memory according to claim 1, wherein,
the content of the component (A) is 0.1 mass % or more and 35 mass % or less.

3. The composition for improving the comprehensive memory according to claim 1, wherein,
the ester body of the astaxanthin is a monoester body and a diester body, and the mass ratio (monoester body/diester body) of the contents of the monoester body to the diester body is 2 or more and 25 or less.

4. The composition for improving the comprehensive memory according to claim 1, wherein,
the composition is prepared to ingest the component (A) in an amount of 3 mg or more and 15 mg or less per day.

5. The composition for improving the comprehensive memory according to claim 1, wherein,
the composition is prepared to ingest the component (A) in an amount of 3 mg or more and 10 mg or less per day.

6. The composition for improving the comprehensive memory according to claim 1, wherein,
the composition is prepared to ingest the component (A) in an amount of 8 mg or more and 10 mg or less per day.

7. The composition for improving the comprehensive memory according to claim 1, wherein,
the composition is prepared to ingest the component (B) in an amount of 30 mg or more and 70 mg or less per day.

8. The composition for improving the comprehensive memory according to claim 1, wherein,
the component (B) is tocotrienols.

9. The composition for improving the comprehensive memory according to claim 1, wherein,
the ingestion is oral ingestion.

10. The composition for improving the comprehensive memory according to claim 1, wherein,
the composition is a food.

11. The composition for improving the comprehensive memory according to claim 1, wherein,
the composition is a food additive.

12. A method for improving comprehensive memory, wherein,
the method comprises ingesting a component (A), or a component (A) and a component (B) to a subject, and the comprehensive memory comprises visual memory and language memory;
the component (A): at least one selected from a group consisting of astaxanthin and an ester body of the astaxanthin, and
the component (B): vitamin E.

13. The method for improving the comprehensive memory according to claim 12, wherein,
the ester body of the astaxanthin is a monoester body and a diester body, and the mass ratio (monoester body/diester body) of the daily intakes of the monoester body to the diester body is 2 or more and 25 or less.

14. The method for improving the comprehensive memory according to claim 12, wherein,
the method comprises ingesting at least one selected from a group consisting of astaxanthin and an ester body of the astaxanthin in an amount of 3 mg or more and 15 mg or less per day.

15. The method for improving the comprehensive memory according to claim 12, wherein,
the method comprises ingesting the component (A) in an amount of 3 mg or more and 10 mg or less per day.

16. The method for improving the comprehensive memory according to claim 12, wherein,
the method comprises ingesting the component (A) in an amount of 8 mg or more and 10 mg or less per day.

17. The method for improving the comprehensive memory according to claim 12, wherein,
the method comprises ingesting the component (B) in an amount of 30 mg or more and 70 mg or less per day.

18. The method for improving the comprehensive memory according to claim 12, wherein,
the component (B) is tocotrienols.

19. The method for improving the comprehensive memory according to claim 12, wherein,
the ingestion is oral ingestion.

20. The method for improving the comprehensive memory according to claim 12, wherein the method comprises ingesting composition to the subject,
wherein the composition comprises the component (A), or a component (A) and a component (B); wherein the composition is a food or a food additive.
